(19) United States
(12) Patent Application Publication
(10) Pub. No.: US 2026/0315006 A1
(43) Pub. Date: Oct. 15, 2026

(54) VEHICULAR OCCUPANT MONITORING SYSTEM WITH OPTICAL SENSOR FOR OCCUPANT HEALTH MONITORING

(71) Applicant: Magna Electronics Inc., Auburn Hills, MI (US)

(72) Inventors: Divya Ramesh, Karnataka (IN); Usham V. Dias, Goa (IN)

(21) Appl. No.: 19/640,760

(22) Filed: Apr. 7, 2026

Related U.S. Application Data
(60) Provisional application No. 63/787,009, filed on Apr. 11, 2025.
(60) Provisional application No. 63/841,448, filed on Jul. 10, 2025.

(51) Int. Cl. A61B 5/00; A61B 5/0205; A61B 5/024; A61B 5/08; A61B 5/103; A61B 5/1171; B60Q 9/00; B60R 1/04; B60R 1/12; B60W 30/06; B60W 50/14; G06T 7/00; G06V 20/59; G06V 40/16

(52) U.S. Cl. CPC A61B 5/6893; A61B 5/0205; A61B 5/1176; B60Q 9/00; B60R 1/12; B60W 30/06; B60W 50/14; G06T 7/0012; G06V 20/593; G06V 40/16; A61B 5/02405; A61B 5/02416; A61B 5/0816; A61B 5/1032; B60R 1/04; B60R 2001/1253; B60W 2420/403; B60W 2540/043; B60W 2540/221; B60W 2556/45; G06T 2207/10024; G06T 2207/10048; G06T 2207/30088; G06T 2207/30201; G06T 2207/30268

(57) ABSTRACT

A vehicular cabin monitoring system includes a camera disposed within an interior cabin of a vehicle. The camera views within the interior cabin. The system, via processing at an electronic control unit (ECU) of image data captured by the camera, monitors an occupant of the vehicle. The system determines a skin tone of the occupant. Based on the determined skin tone of the occupant of the vehicle, and responsive to detecting a change in complexion for the occupant of the vehicle, the system determines a health condition for the occupant of the vehicle.

### CROSS REFERENCE TO RELATED APPLICATIONS

The present application claims the filing benefits of U.S. provisional application Ser. No. 63/841,448, filed Jul. 10, 2025, and U.S. provisional application Ser. No. 63/787,009, filed Apr. 11, 2025, which are hereby incorporated herein by reference in their entireties.

### FIELD OF THE INVENTION

The present invention relates generally to a vehicular cabin monitoring system for a vehicle and, more particularly, to a vehicular cabin monitoring system that utilizes one or more sensors at a vehicle.

### BACKGROUND OF THE INVENTION

Use of imaging sensors in vehicle imaging systems is common and known. Examples of such known systems are described in U.S. Pat. Nos. 5,949,331; 5,670,935 and/or 5,550,677, which are hereby incorporated herein by reference in their entireties.

### SUMMARY OF THE INVENTION

An example vehicular cabin monitoring system includes a camera disposed within an interior cabin of a vehicle equipped with the vehicular cabin monitoring system. The camera views within the interior cabin of the vehicle. Image data captured by the camera is transferred to an electronic control unit (ECU). The ECU includes electronic circuitry and associated software, and the electronic circuitry of the ECU includes an image processor operable to process image data transferred to the ECU. The vehicular cabin monitoring system, via processing at the ECU of image data captured by the camera, monitors a driver and/or occupant of the vehicle within the interior cabin of the vehicle. Responsive to detecting a change in facial complexion for the driver and/or occupant of the vehicle, the vehicular cabin monitoring system determines a health condition for the driver and/or occupant of the vehicle, such as a heart rate, a heart rate variability, and/or a breathing rate. The system may generate an alert responsive to the health condition being indicative of a poor health condition for the driver and/or occupant of the vehicle.

These and other objects, advantages, purposes and features of the present invention will become apparent upon review of the following specification in conjunction with the drawings.

### BRIEF DESCRIPTION OF THE DRAWINGS

FIG. 1 is a side view of a vehicle having a vehicular cabin monitoring system;

FIG. 2 is a schematic view of optical heart rate sensing;

FIG. 3 shows the details of a Butterworth Bandpass filter of the 186th order;

FIG. 4 shows the details of a Butterworth Bandpass filter of the 6th order;

FIG. 5 is a schematic view of the vehicular cabin monitoring system;

FIG. 6 is a flowchart of an example method of determining a vital sign of an occupant of the vehicle based on a determined skin tone of the occupant and a determined change in complexion;

FIG. 7A is a schematic view of a single Bandpass filter for estimating heart rate and breathing rate together;

FIG. 7B is a schematic view of separate Bandpass filters for separately estimating heart rate and breathing rate;

FIG. 8 is a diagram of different regions of interest of an occupant for determining skin tone and changes in complexion;

FIG. 9 shows an example output of a processed frame of image data from a publicly available dataset for image segmentation and skin color detection;

FIG. 10 shows example raw and filtered signals for the R component, with a window size of 60 and using the filter of FIG. 4;

FIG. 11 shows example raw and filtered signals for the G component, with a window size of 60 and using the filter of FIG. 4;

FIG. 12 shows example raw and filtered signals for the B component, with a window size of 60 and using the filter of FIG. 4;

FIG. 13 shows example raw and filtered signals for the GR component, with a window size of 60 and using the filter of FIG. 4;

FIG. 14 shows example raw and filtered signals for the GB component, with a window size of 60 and using the filter of FIG. 4;

FIG. 15 shows example raw and filtered signals for the GRGB component, with a window size of 60 and using the filter of FIG. 4;

FIG. 16 shows example raw and filtered signals for the R component, with a window size of 128 and using the filter of FIG. 4;

FIG. 17 shows example raw and filtered signals for the G component, with a window size of 128 and using the filter of FIG. 4;

FIG. 18 shows example raw and filtered signals for the B component, with a window size of 128 and using the filter of FIG. 4;

FIG. 19 shows example raw and filtered signals for the GR component, with a window size of 128 and using the filter of FIG. 4;

FIG. 20 shows example raw and filtered signals for the GB component, with a window size of 128 and using the filter of FIG. 4;

FIG. 21 shows example raw and filtered signals for the GRGB component, with a window size of 128 and using the filter of FIG. 4;

FIG. 22 shows example heart rate estimation for the signals with a window size of 60;

FIG. 23 shows example heart rate variability estimation for the signals with a window size of 60;

FIG. 24 shows example heart rate estimation based on the G component using a filter of the 6th order and a window size of 128;

FIG. 25 shows example heart rate variability estimation based on the G component using a filter of the 6th order and a window size of 128;

FIG. 26 is a table that includes mean square error (MSE) values for heart rate estimation for the signals with a 6th order filter and a window size of 60;

FIG. 27 is a table that includes MSE values for heart rate variability estimation for the signals with a 6th order filter and a window size of 60;

FIG. 28 is a table that includes mean square error (MSE) values for heart rate estimation for the signals with a 186th order filter and a window size of 60;

FIG. 29 is a table that includes MSE values for heart rate variability estimation for the signals with a 186th order filter and a window size of 60;

FIG. 30 is a table that includes MSE values for heart rate estimation for the signals with a 6th order filter and a window size of 128;

FIG. 31 is a table that includes MSE values for heart rate variability estimation for the signals with a 6th order filter and a window size of 128;

FIG. 32 is a table that includes MSE values for heart rate estimation for the signals with a 186th order filter and a window size of 128;

FIG. 33 is a table that includes MSE values for heart rate variability estimation for the signals with a 186th order filter and a window size of 128; and

FIG. 34 is a table that includes average MSE values for heart rate and heart rate variability estimation across all videos with different filter orders and window sizes.

### DESCRIPTION OF THE PREFERRED EMBODIMENTS

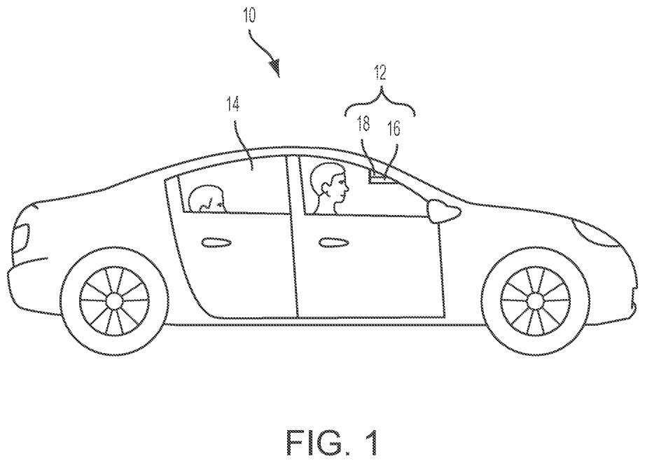

Referring now to the drawings and the illustrative embodiments depicted therein, a vehicular cabin monitoring system 12 for a vehicle 10 is configured to detect presence of occupants within the interior cabin 14 of the vehicle 10 (FIG. 1). For example, the system 12 may include one or more sensors 16 disposed at the interior cabin 14 of the vehicle 10 and that sense respective regions of the interior cabin 14 of the vehicle 10. For example, the sensor may include a radar sensor, a camera, an optical sensor, and the like. The system 12 further includes a control or electronic control unit (ECU) 18 that includes electronic circuitry and associated software, such as a data processor or image processor for processing sensor data captured by the one or more sensors 16, whereby the ECU 18 may process sensor data captured by the one or more sensors 16 to detect presence of occupants, determine conditions of the occupants, and the like, as discussed further below. Although shown as disposed at an overhead console module of the vehicle 10, it should be understood that the one or more sensors 16 and/or the ECU 18 may be disposed at any suitable portion of the interior cabin 14 of the vehicle 10 for sensing regions of the interior cabin 14, such as at an interior rearview mirror assembly, a light module (e.g., a reading light or dome light), a grab handle module, at or behind the vehicle headliner, at the dashboard, and/or at or near the steering wheel of the vehicle. The data transfer or signal communication from the one or more sensors 16 to the ECU 18 may comprise any suitable data or communication link, such as a vehicle network bus or the like of the equipped vehicle.

Remote photoplethysmography (rPPG) may be used to measure the vital signs of a person, such as heart rate, heart rate variability and breathing rate. This enables the ability to remotely monitor a patient. This technology may be used to estimate the driver vitals, such as using driver monitoring cameras. This will keep the cardiovascular health of the driver on check while driving, such as to avoid accidents, injuries to passersby and loss of public property. In addition, serious levels of stress could be monitored based on the irregularities in the heart rate.

This rPPG algorithm through driver monitoring cameras may estimate driver vitals through the measure of reflectance of various color components of the light incident on the driver's face. The rPPG method may utilize the driver monitoring systems available to allow the vehicle to make decisions—either an alarm can go on (to alert the driver in cases of stress or the passersby in case of illness of driver) or the vehicle is safely parked depending on the level of autonomy the vehicle has. This also does not cause any discomfort or distractions to the driver as it is remotely sensed. Since this is not built on heavy algorithms and uses the basic principle of reflectance of different color waves from the source, it is cost effective.

The driver monitoring comes with challenges, such as the illumination of driver's face, the head motion and various skin that tones must be considered by the system.

This can be built by taking real driving scenarios from an in-house camera placed in the car facing the driver. That is, the algorithm may be trained based on image data captured by the driver monitoring or occupant monitoring camera within the cabin of the vehicle during real driving scenarios. The algorithm may be trained prior to release of the system to the customer (e.g., so that each vehicle equipped with the cabin monitoring system is powered by an algorithm trained on the same training data) or the algorithm may be trained during use of the vehicle by the customer (e.g., so that the cabin monitoring system at the vehicle is trained based on image data of the one or more drivers that use the vehicle).

The estimated vital signs from this algorithm can be evaluated against a ground truth like vital sign measurement through oximeter. The video streaming can be taken and checked for the various conditions, such as rest—when the driver is steadily driving, head movements—when the driver looks away from the camera to look around such that a front view of the face cannot be captured, head rotation—the driver rotates his head completely such that only the side view of his face is visible, multi-person scenario—when any of the fellow passenger's face overlaps with the driver causing occlusions or background noise, lighting variations (natural and artificial)—due to change in environment during the day, infrared light—to detect during the night as RGB components cannot be used to determine the vitals, and skin tones, ethnicity and facial structures—to be inclusive for optimized skin detection due to the diversity of human faces. These given conditions can be used to provide datasets to the algorithm. This helps in optimising the rPPG algorithms for real time driving.

The Red, Blue and Green signals are filtered to remove any other noise, and the sliding window approach is used to measure the vital signs from these RGB signals. The sliding window helps to achieve a more realistic transition as it follows the principle of First in First out. The oldest value of the signal over time is pushed out and the next value of the signal is considered. This ensures a smooth transition and avoids sudden difference in the measurement of the estimated heart rate. The ratios of Green to Red and Green to Blue and the sum of these ratios is also considered as part of the algorithm to determine which method works best. Beside these specific ratios, other mathematical formulations or transforms may be applied on the Red, Green and Blue signals. There are other methods that can be used too. The system thus may utilize sensors, such as radar sensors or imaging radar sensors, to detect presence of and/or range to objects.

As discussed further below, the cabin monitoring system processes image data captured by one or more cabin monitoring cameras, such as a driver monitoring camera disposed at the interior rearview mirror assembly of the vehicle, to determine a vital sign of the driver and/or one or more occupants of the vehicle. For example, the system may determine heart rate, heart rate variability, and a breathing rate of the driver and/or occupant or passenger. The system determines the vital sign of the driver based at least in part on detecting changes in facial complexion of the driver. The system detects changes in facial complexion by monitoring red, green, and blue signals of portions of the image data representative of the driver's face and determining changes over time in the ratios between the red, green, and blue signals. The system may have control mechanisms to detect and/or adapt for head movement, differences in skin tone, changes in ambient light, and may monitor portions of the driver's face most likely to exhibit changes in complexion representative of a change in vital sign.

Remote photoplethysmography (rPPG) is a contactless method for estimating vital signs such as heart rate and heart rate variability by analyzing changes in skin-reflected RGB components due to blood volume fluctuations. The cabin monitoring system provides an rPPG-based system for driver health monitoring, addressing the challenges of using conventional pulse oximeters and ECG in a moving vehicle. By integrating rPPG into driver monitoring systems, real-time health assessments can be conducted to prevent accidents caused by sudden cardiac events or high stress levels. The system may use state-of-the-art algorithms, implement cost-effective solutions, and assesses accuracy against ground truth measurements.

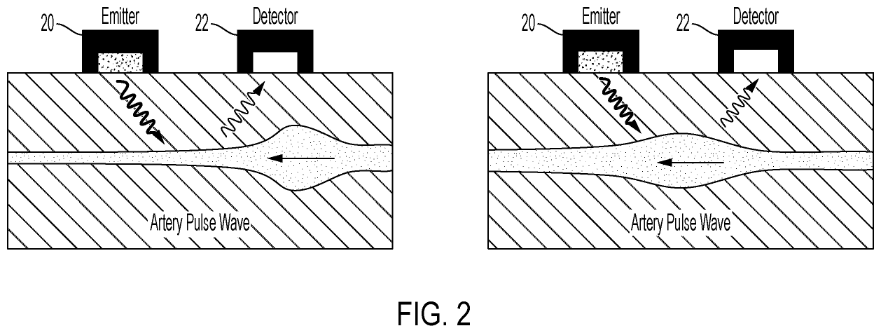

The rPPG method is based on the changes in the RGB components reflected by the skin when blood volume in the skin changes. The color variations are captured by the camera 16 over time and processed to detect the heart rate and heart rate variability of the driver and/or one or more occupants of the vehicle. FIG. 2 illustrates optical heart rate sensing. The left image shows lower pressure preceding the pulse wave which means narrower arteries and less absorption (higher reflectivity) of the light source while the right image shows a higher blood pressure pulse that causes wider arteries and more light absorption (lower reflectivity). In other words, light emitted by a light source 20 and reflected back to a detector 22 may be higher or increase during low pressure portions or lower blood volume portions of a heart beat as compared to high pressure portions or higher blood volume portions of the heartbeat.

Vital sign estimation, such as, for example, breathing rate, heart rate and heart rate variability estimation, is essential to monitor and detect potential health issues quickly. Major challenges have been noted while monitoring the health of the driver in a moving vehicle since using a pulse oximeter and ECG is not possible in such a situation. The cabin monitoring system 12 uses rPPG for vital sign estimation. The rPPG method is based on the changes in the RGB components reflected by the skin when blood volume in the skin changes. The color variations are captured by the camera 16 over time and processed to detect the heart rate and heart rate variability. Therefore, this method is contactless and would not cause any discomfort to the driver. The system 12 may implement a simple solution or a complex algorithm may be implemented in a cost-effective manner. The accuracy of vital sign estimation may be evaluated with respect to the ground truth.

A study of forensic pathological anatomic reports of sudden death (ischemic heart disease) while driving showed that 2 out of 21 drivers complained of discomfort or nausea before death while the other 19 drivers did not have any symptoms or were ignorant of the mild symptoms. This may indicate a need to monitor the driver's vitals such as heart rate and heart rate variability on a constant basis while driving to avoid accidents, injuries to passersby and loss of public property. In addition, serious levels of stress could be monitored based on the irregularities in the heart rate. The rPPG method may be integrated into the driver monitoring systems to allow the vehicle to make decisions either an alarm can go on (to alert the driver in cases of stress or the passersby in case of illness of driver) or the vehicle is safely parked depending on the level of autonomy the vehicle has. This also doesn't cause any discomfort or distractions to the driver as it is remotely sensed.

The rPPG signals may be extracted using RGB video signals. For example, a low-complexity algorithm may eliminate the need for machine learning or complex computational models. The method employs a combination of green-to-red (GR) and green-to-blue (GB) ratios and utilizes RGB signals extracted from specific facial regions, such as the forehead and cheeks. Evaluations conducted on the local group invariance for heart rate estimation from face videos in the wild (LGI-PPGI) dataset, which includes diverse scenarios like resting, gym activities, talking, and head rotations, demonstrated that this approach outperformed machine-learning-based rPPG methods. The algorithm may be robust against variations in indoor lighting and participant movement, offering superior heart rate estimation. Thus, remote cardiovascular health monitoring may be achieved using consumer-grade cameras.

The system may utilize the mathematical principles underlying rPPG pulse extraction and provide a robust algorithm for improved performance. The system may use a skin reflection model that integrates physiological and optical properties. The system may further use PCA/ICA (Blind Source Separation), CHROM, PBV, and Plane-Orthogonal-to-Skin (POS). Among these, the POS method may provide a more motion-robust approach, as it utilizes projections orthogonal to skin tone. The system may demonstrate that POS achieves reliable pulse extraction, particularly in motion-heavy environments.

The system may utilize a standardized framework for evaluating rPPG algorithms with statistical rigor. For example, pyVHR, an open-source Python package designed to assess rPPG algorithms comprehensively, may be used. Five datasets, including PURE, LGI, UBFC, MAHNOB, and COHFACE, may be utilized. By employing nonparametric statistical tests, the system may ensure fair comparisons among competing algorithms. Top-performing methods such as POS, CHROM, PCA, and SSR may not exhibit significant statistical differences in accuracy. The system may use standardized preprocessing and evaluation techniques to enhance the credibility of rPPG.

The system may use a motion-robust rPPG method capable of dynamically adjusting the regions of interest (ROIs) on the face to enhance monitoring accuracy. Facial landmark detection techniques may be employed to adaptively track ROIs during movement. The periodic components of RGB signals may be extracted to estimate heart rate, and validation may be performed against skin-contact PPG sensors. The adaptive ROI approach may successfully maintain signal stability during head movements. A high correlation may be present between the estimated heart rates and the ground truth.

The system may consider how facial makeup affects the quality of rPPG signals in both visible (RGB) and near-infrared (NIR) light. The system may measure pulse-rate strength using the AC/DC ratio and analyze PPG signature changes across different wavelengths. Makeup may reduce light penetration, leading to lower signal quality in RGB-based rPPG methods. NIR-based rPPG may be less affected by makeup.

The system may utilize rPPG in near-infrared (NIR) light for enhanced robustness against motion artifacts. The system may use single-camera setups (RGB with an NIR filter) and/or multi-camera configurations. Additionally, spectrally optimized LED illumination may be used to improve motion resilience. Experiments conducted on a dataset comprising 30 videos of individuals with varying skin tones and motion conditions demonstrated that both setups provided accurate pulse extraction. Notably, single-camera setups performed better in scenarios involving scaling motions. The findings highlight the potential of NIR-based rPPG for monitoring vital signs in low-light conditions and high-motion environments, such as fitness and driving applications.

The system may analyze computationally efficient facial regions for extracting high-quality rPPG signals while minimizing processing complexity. The system may divide the face into seven regions, including the forehead, cheeks, nose, nasion, mouth, and chin, and these regions may be compared based on their signal quality based on signal-to-noise ratio (SNR) and correlation coefficient (CC) with reference PPG. The forehead and cheeks may provide the most reliable signals, yielding high SNR and CC values. In contrast, the mouth and chin may exhibit poor signal quality due to muscle movements and facial hair, while the nasion and nose may produce inconsistent results.

The UBFC-rPPG dataset may be used to evaluate rPPG methods by providing synchronized facial video recordings along with contact-based PPG signals serving as ground truth. The dataset comprises videos of subjects engaged in various tasks while seated in a controlled environment. Ground truth PPG signals are collected using a fingertip pulse oximeter, and RGB video recordings are captured at a resolution of 640×480 pixels with a frame rate of 30 frames per second. The dataset includes steady-condition videos to minimize external variations such as motion or lighting, ensuring that participants remain relatively still. This controlled setup facilitates benchmarking of rPPG algorithms. The dataset proves valuable for testing heart rate estimation algorithms, assessing the robustness of rPPG against motion and lighting changes, and developing contactless physiological monitoring methods. Advantages of the UBFC-rPPG dataset include the availability of ground truth PPG signals for comparison, suitability for low-noise algorithm testing, and its use in rPPG research, allowing for fair comparisons among methods. However, its limitations include restricted diversity in motion scenarios and environmental variations, necessitating additional datasets for testing robustness under uncontrolled conditions.

The Public Benchmark Dataset for Testing rPPG Algorithm Performance provides a standardized dataset aimed at evaluating the accuracy and robustness of rPPG algorithms under diverse conditions, including motion and varying lighting environments. It contains facial video recordings under both controlled and semi-controlled scenarios, with ground truth physiological signals such as heart rate provided for reference. The dataset features multiple scenarios, including stationary conditions where subjects remain seated with minimal movement, simulated motion activities like head movements, and variations in lighting, including both natural and artificial sources. High-resolution video recordings ensure sufficient detail for rPPG signal extraction, making them suitable for testing ROI-based and pixel-level processing methods. Additionally, ground truth PPG signals are captured using standard contact devices like ECG, ensuring precise temporal alignment with video frames. This dataset is particularly useful for benchmarking rPPG algorithms against motion artifacts, evaluating heart rate and heart rate variability estimation techniques, and developing adaptive rPPG methods for real-world applications. Its primary advantages lie in its standardized setup, which ensures result reproducibility, its diverse scenarios that simulate real-world rPPG conditions, and its ability to facilitate algorithm comparison. However, its limitations include its predefined scenarios, which may not cover extreme cases such as intense physical activity or very low-light conditions, making integration with additional datasets necessary for specific applications like neonatal monitoring or multi-person detection.

The MPSC-rPPG dataset (Multi-Person Scenario Collection) is designed to evaluate rPPG algorithms in multi-person settings with challenging conditions, including variations in lighting, motion, and occlusions. The dataset contains high-definition videos of multiple individuals captured in both indoor and outdoor environments, simulating real-world activities such as conversations and interactive engagements. Ground truth physiological signals are recorded for each individual using contact-based PPG devices, ensuring precise synchronization with the corresponding video frames for accurate benchmarking. The dataset is characterized by diverse environmental variations, including different lighting conditions (natural and artificial) and various motion scenarios such as head and body movements. Its features include the multiperson setup, which enables testing of algorithms for distinguishing and extracting signals from multiple individuals simultaneously, as well as the inclusion of occlusions, background noise, and dynamic lighting to simulate real-world challenges. Additionally, comprehensive metadata annotations provide details on individual identities, motion types, and environmental settings. The dataset supports the development and testing of rPPG algorithms for applications such as group heart rate monitoring, surveillance, fitness tracking, and telemedicine. Its advantages include providing a unique multi-person setup lacking in many existing datasets, incorporating realistic environmental variations for testing algorithm robustness, and supporting adaptive methods for handling occlusions and complex scenarios. However, the dataset presents challenges in terms of increased data complexity, requiring advanced preprocessing and machine learning techniques to effectively extract individual signals, and difficulties in signal extraction due to overlapping facial regions and occlusions.

The rPPG Avatars Dataset offers a synthetic, highly controllable environment for testing rPPG algorithms under various simulated conditions, ensuring reproducibility and detailed parameter control. The dataset comprises computer-generated avatars designed to mimic human faces, incorporating pulsatile skin reflectance patterns that correspond to heartbeat activity. The synthetic videos include a variety of scenarios, such as head movements, facial expressions, and changes in lighting conditions, to simulate real-world applications. Additionally, perfectly synchronized ground truth PPG signals are generated with minimal noise, establishing a direct correlation between skin reflectance changes and simulated pulse waveforms. The dataset is highly customizable, allowing modifications to avatar appearance, such as skin tone and facial features, and environmental factors like lighting, camera angle, and motion. An advantage of this dataset is its ability to simulate the physiological basis of rPPG with virtual heartbeats while providing a controlled environment free from external noise, allowing for isolated testing of motion, lighting, and other variables. The dataset also includes diverse avatars representing different ethnicities, skin tones, and facial structures, enhancing its usability for various applications. The dataset may be suitable for benchmarking rPPG algorithms under completely reproducible conditions, evaluating performance across diverse simulated scenarios, and analyzing the impact of specific factors such as motion and lighting on rPPG signal quality. However, its limitations stem from its synthetic nature, as it lacks the complexities of real-world skin reflectance, including subtle variations in blood perfusion. Additionally, it does not directly represent environmental artifacts such as ambient noise or extreme lighting conditions, which could impact real-world performance.

The Facial Video-Based Heart Rate Monitoring Dataset was developed to provide a dedicated dataset for testing heart rate monitoring algorithms that rely on facial video data. This dataset includes high-definition video recordings of subjects captured under controlled and semi-controlled conditions, incorporating variations in motion, lighting, and facial expressions. The dataset includes synchronized ground truth signals collected using contact-based devices such as pulse oximeters, ensuring accurate benchmarking. The diverse scenarios included in the dataset feature recordings under multiple lighting conditions, both natural and artificial, as well as various head movement and motion scenarios to test the robustness of rPPG algorithms. The dataset is valuable for evaluating data quality, as its high-resolution videos enable precise extraction of facial features necessary for accurate signal analysis. Additionally, it provides controlled variability in terms of skin tones, facial structures, and environmental settings, making it useful for studying the impact of these factors on algorithm performance. The dataset serves as a strong benchmark for comparing facial video-based heart rate monitoring algorithms, enabling fair and reproducible evaluations. Its advantages include the availability of synchronized ground truth data, the inclusion of diverse conditions to test algorithm adaptability, and its support for reproducible research. However, its limitations include a lack of extreme real-world scenarios such as intense motion or very low-light conditions. In such cases, additional datasets may be required to supplement its applications in specialized areas like neonatal monitoring or multi-person detection.

The Public Benchmark Dataset for Testing rPPG Algorithm Performance may be utilized by the system due to its easy accessibility and inclusion of the ground truth. The dataset includes 21 RGB videos in the . avi file format which address the following four challenging factors; Lighting-, skin tone-, motion-, and high heart rate robustness. These 21 individual files hold both the raw and synchronized data. The frame rate of these video files is 30 frames per second. The ground truth provided for this data set is the ECG RR interval information files.

The RGB video files were loaded to the MATLAB™ VideoReader function. The video frames were resized to ensure uniform dimensions for consistent processing. The extracted individual frames from the video were further analyzed and a vision. CascadeObjectDetector( ) function was used to detect faces in the frames. The face location is obtained as a rectangular region defined by x-coordinate, y-coordinate, width, and height. The detected face region is cropped based on the coordinates and metrics from this function to isolate the region of interest (e.g., the complete human face region).

The cropped face image from the face Segmentation is converted to HSV color space from the RGB color space. The Hue, Saturation, and Value are extracted as individual components. Hue stands for the color type which is represented as an angle on a color wheel, such that red is 0 degrees, green is 120 degrees, and blue is 240 degrees. Saturation gives the measure of the vibrancy of the color, represented as a percentage, such that 0 percent is a shade of gray and 100 percent is the full color. Value is a measure of the brightness of the color, represented as a percentage, such that 0 percent is completely dark and 100 percent is fully bright. For skin pixel detection, thresholds are applied to detect skin regions, hue ranges are between 0 and 0.1 as the human skin tone is more towards the orange tone. This is inclusive of all skin tones. The saturation ranges between 0.2 and 0.6 as very low saturation values indicate desaturated colors or grayscale which are not reliable for skin detection and very high saturation values capture unnatural lighting effects or strong shadows. These ranges for skin detection have been taken based on the MATLAB™ tutorial. However, the thresholds may be designed as calibratable or adaptive parameters. For example, depending on the lighting condition or specific occupant skin tones. A binary mask is used to differentiate between skin pixels and non-skin pixels that would be used for further processing. Mask values of 1 for skin pixels and 0 for non-skin pixels which on the human face would include the human facial hair pixels like the eyebrow, beard, moustache and also the eye region on the face.

The average of the RGB components of the skin pixels may been taken from the original face detected cropped image using the binary skin mask pixel locations. The individual R, G, B components are extracted, in addition to this the ratios of Green to blue (GB), Green to Red (GR) and the sum of these ratios (GRGB). These six signal values are further processed using filters.

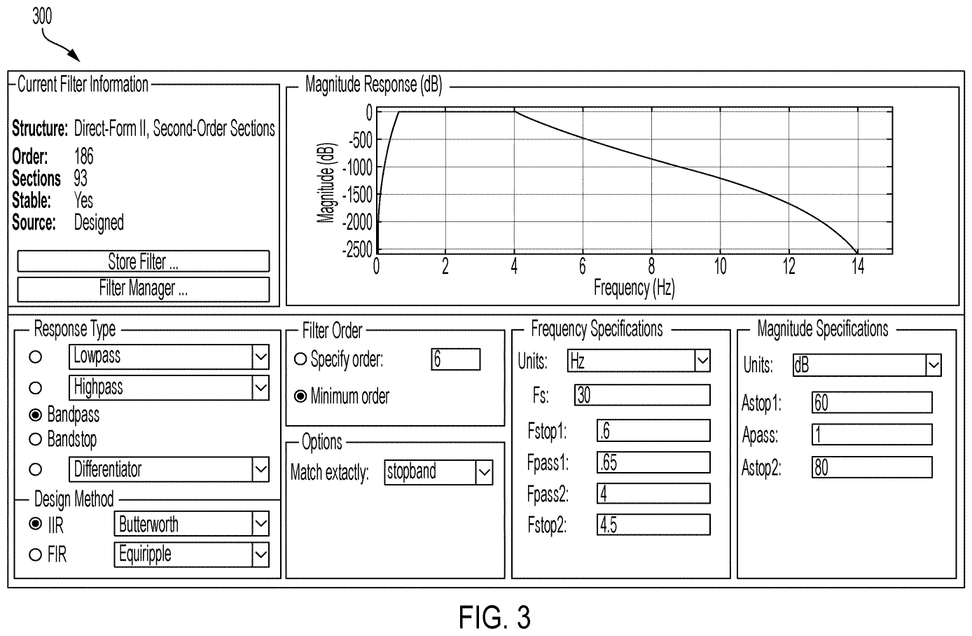

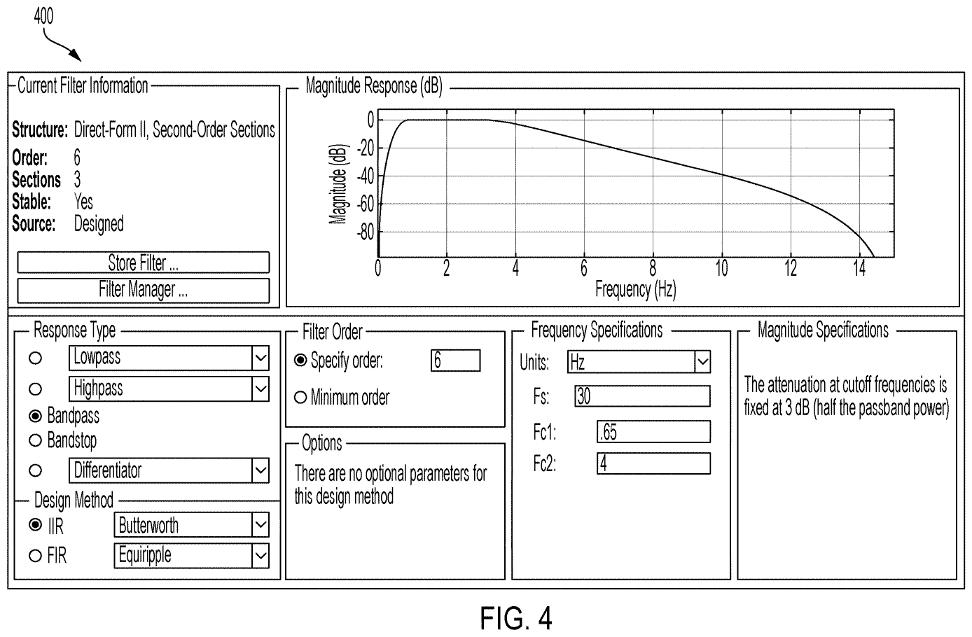

The six signals of R, G, B, GR, GB, GRGB are passed through a bandpass butterworth filter to filter out any noise in the raw signals. In general, the normal human heart rate is about 60 bpm to about 100 bpm and it varies based on age and health conditions. Any filter design needs to take into account the frequencies of interest. For heart rate estimation the frequencies range from 0.6 Hz to 4 Hz. A bandpass filter is designed in MATLAB™ using the filter designer application. In one case the default minimum order filter was considered which generated a 186th Order IIR Butterworth Bandpass filter. Refer to the schematic 300 of FIG. 3 for the design details. Another filter of Order 6 (low complexity implementation) was designed, shown by the schematic 400 in FIG. 4. In general, any IIR or FIR filter may be designed based on the requirements/constraints.

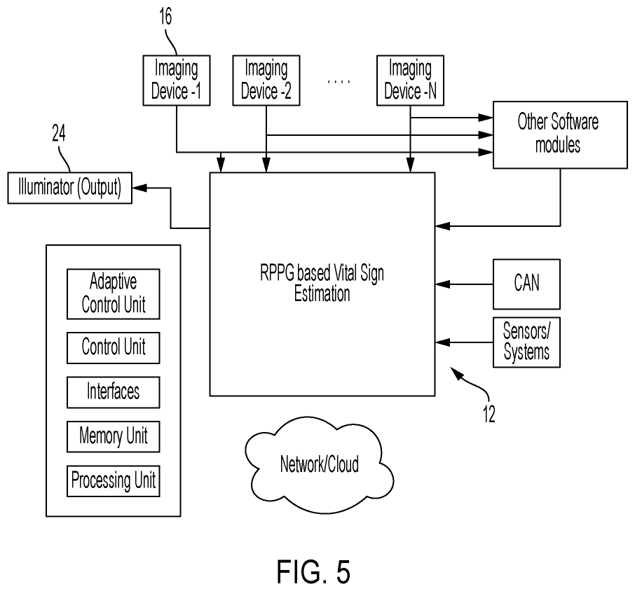

Referring to FIG. 5, the vehicular cabin monitoring system 12 receives sensor data from one or more sensors or imaging devices 16. The imaging device 16 may be configured to capture sensor data representative of any suitable frequency range, such as visible light, infrared or near infrared light, and the like. In some examples, there may be multiple imaging devices 16 of different types. Further, the imaging device 16 may include additional features, such a time-of-flight (ToF) option, and/or the imaging device 16 may include or be associated with an illuminator or light source 24. Communication between the imaging device 16 and the system 12 may be wired or wireless.

The illuminator or light source 24 may include a visible light source and/or an infrared or near infrared light source operable to emit infrared or near infrared light that illuminates at least a portion of the interior cabin of the vehicle. Thus, the light source 24 may emit light in green, red, blue and/or IR wavelengths/frequencies and the imaging device 16 receives the reflected signals to estimate vital signs.

That is, the system 12 processes images input from the imaging device 16 to estimate the vital signs of the driver and/or occupant or passenger of the vehicle. For example, the system 12 determines heart rate, heart rate variability, breathing rate, and the like. The system 12 may use inputs from other available sensors, modules, software, units, and the like, to adapt parameters, modify features, modify data, modify estimates, and the like. Examples of performance parameters may be false positives, accuracy, and the like. Complexity parameters may include computational complexity like addition, multiplication, power consumption, memory utilization, and the like.

For example, the system 12 may receive signals from other sensors or systems of the vehicle, such as a radar sensor or system or intelligent clearance sonar (ICS) to determine occupant location, classification information, and the like. Further, the system 12 may receive signals and/or sensor data from one or more of a seat pressure sensor, a seat belt detector, a vibration or motion sensor, a direct or indirect ToF sensor, and the like.

Some sensors may be integrated together and/or to the camera system. The system 12 may include software and associated outputs for processing sensor data and/or transmitting commands to the sensors or systems of the vehicle.

Moreover, the system 12 may communicate via the vehicle CAN bus. For example, a signal over the CAN bus indicating that the door of the vehicle is opened or closed may be used to reset or modify adaptive parameters.

All sensor modules may have the necessary control units, interfaces, associated software, memory unit, processing unit, and the like. These may be shared across modules, sensors, and software, including the rPPG system. Further, the sensor modules may include adaptive control. For example, the light source may be adaptively controlled. The system 12 internally may include separate adaptive control for estimating methods, process, parameters, and the like. The system 12 may have access to a network or cloud storage. The system 12 may have two way communication with other sensors, systems, modules, software, and the like. For example, the system 12 may provide outputs to existing driver monitoring systems or interior cabin monitoring systems.

Optionally, a user profile may be stored in memory (e.g., cloud storage or at the vehicle) for one or more individual occupants and/or drivers of the vehicle, and the user profile may be used for determining the skin tone and/or a change in complexion for the occupant and/or driver of the vehicle. For example, the user profile may include demographic details corresponding to the user, such as an ethnicity, gender, age and the like, which may include an average skin tone or skin pixel value for the user. Thus, changes in complexion for the user may be determined based on changes in pixel value from the stored skin tone and/or from an expected skin tone determined based on the stored skin tone. The user profile may be retrieved from storage automatically (e.g., based on identifying the driver and/or occupant) or manually (e.g., based on the driver and/or occupant selecting their user profile during operation of the vehicle).

In some examples, the user profile may include information related to a health condition or health history of the user. For example, the profile may include average changes in skin tone or complexion for the user so that an alert may be generated if the complexion of the user is determined to be outside of an expected range. Determinations of heart rate, heart rate variability, and breathing rate may be added to the stored profile for future determinations.

The system may determine the health condition of the occupant automatically or continuously throughout operation of the vehicle (e.g., at regular intervals like every 10 seconds, every 30 seconds, every 1 minute, every 5 minutes, and the like) or the system may determine the health condition of the occupant in response to an input or determined vehicle condition. For example, the system 12 may determine the health condition of the occupant in response to determining that the occupant has entered the vehicle, in response to the vehicle being started or turned on, or based on determination of unusual or atypical vehicle dynamics. These may include determination that the vehicle has been involved in a collision or impact, determination that the vehicle has stopped suddenly, determination that the vehicle has steered or swerved suddenly, determination that the vehicle has accelerated suddenly, and/or determination that the vehicle is being operated differently from other vehicles surrounding the vehicle.

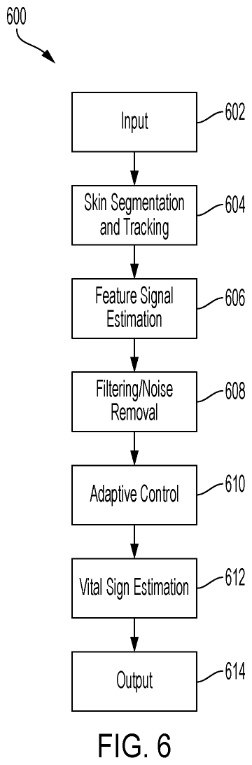

FIG. 6 shows a flowchart for an example method 600 of estimating vital signs for a driver or occupant of the vehicle 10 based on processing of image data captured by the interior viewing camera 16. At operation 602, the method 600 includes receiving sensor data from at least one imaging device 16. At operation 604, the method 600 includes performing skin segmentation and tracking. The system 12 may identify regions of interest (ROIs) for at least one occupant, subject, person, or user. The system may use apriori knowledge, such as seat of locations in the image. Further, the system may track all skin pixels at a global level for the user, irrespective of whether the pixel is representative of the user's face, neck, hand, and the like. In some examples, the system may use skin pixels from a particular body part, such as only face pixels, only neck pixels, only face-neck pixels, only hand pixels, and the like. The system may track the ROI over time/frames for each occupant. Further, the system 12 may utilize necessary noise, artifact, motion, and illumination estimation and/or compensation.

At operation 606, the method 600 performs feature signal estimation. That is, the system 12 estimates at least one feature value for at least one of the ROI. The feature value may include at least one formula/statistical parameter (e.g., mean or average, standard deviation, and the like). The feature value is determined for at least one channel or wavelength. For example, the average of skin pixel intensity in the ROI (e.g., the skin tone of the occupant) is determined for at least one of Red, Green, and Blue channel values (or any other wavelength such as IR wavelengths greater than around 700 nm such as wavelengths between 780 nm and 1,000 nm or thereabouts). Necessary noise removal/filtering may be performed. The channel/wavelength based features may be combined through any formulation to create a new/transformed feature value. For example, the transformed value may be additive, multiplicative, or a ratio of one or more of the Red, Green, and Blue channels. These feature values are stacked and/or combined over time to create a time signal which is expected to hold the vital signs information. Put another way, this feature value may be tracked as the skin tone or a baseline pixel value for skin of the occupant so that changes in complexion or pixel value may be identified for vital sign estimation.

At operation 608, the method 600 performs filtering or noise removal. Although noise removal is illustrated as a separate step in the method 600, noise estimation, removal, and/or compensation may be performed as part of other steps in the method 600. One example of noise removal is finding mean or median values. Other examples of noise removal may include estimating illumination changes, motion, and/or body movement and then compensating for these variations/artifacts. Filtering may also be a kind of noise removal as unwanted signals are removed before continuing to the next processing stage. For example, filtering may include using a band pass filter to pass only the valid heart rate frequencies for heart-rate estimation (and estimation of associated vitals, e.g. heart rate variability). Noise sources may be from inside the vehicle, such as body motion, facial expressions, vibrations, and the like and/or noise sources may be from outside the vehicle, such as light variations due to other vehicles, streetlight, sunlight, external reflected light, glare, and the like. In general, noise may also relate to camera noise, quantization noise, image reconstruction noise, transmission related noise, and the like. Noise may be estimated and then compensated spatially, temporally, across wavelengths, and the like.

At operation 610, the method 600 performs adaptive control by changing parameters, threshold, methods, procedures, and the like based on inputs from any module, software, sensor, and the like internal or external to the rPPG estimation module. In other words, based on the determined skin tone (e.g., an average pixel value for a ROI of the driver or occupant), the system 12 adjusts parameters for determining vital signs of the driver or occupant. For example, the system 12 may adjust buffer size for vital sign estimation, signals of interest, ROIs, a transformation formula, vital sign estimation method, illumination compensation, noise compensation, motion compensation, and the like.

At operation 612, the method 600 includes performing vital sign estimation, such as determining heart rate, heart rate variability, breathing rate, and the like. Time-domain methods may be used for vital sign estimation, such as determining peak difference or using frequency domain methods like Fourier transforms. Vital sign estimation may be performed for each driver and/or occupant for at least one ROI and for at least one feature signal (normal or transformed). If multiple estimates are determined for one occupant, then they may be combined to give a final estimate for the occupant. Combining may involve multiple ROIs, feature signals, imaging devices, and the like. At operation 614, the method 600 provides an output, such as a value for the determined vital sign and/or a determination that the vital sign is indicative of a medical emergency for the driver or occupant.

In general, the method 600 may be performed in any suitable order. For example, certain parts may be included in each step, such as noise removal. The method 600 may be performed to determine vital signs for any suitable number of occupants of the vehicle. Further, a control unit may determine an occupant of interest and/or it may be predefined, user defined, or automatic.

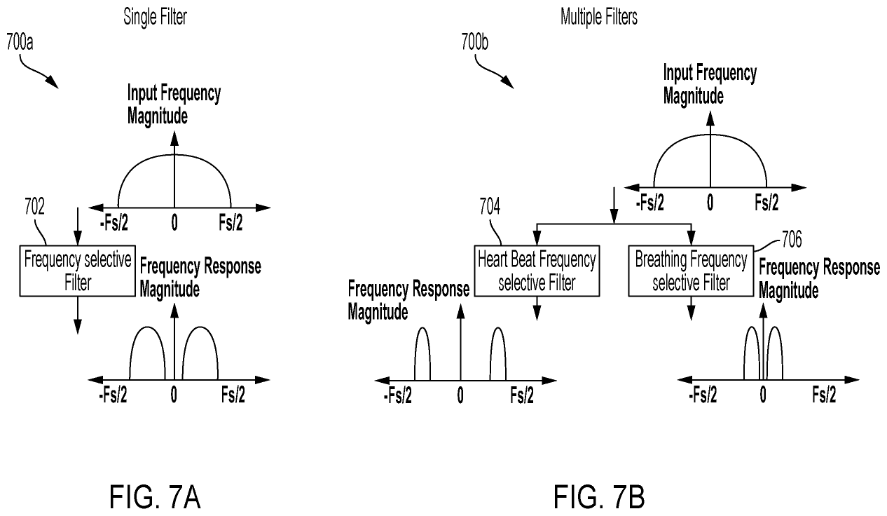

FIGS. 7A and 7B illustrate a bandpass filter for estimation of heart rate and/or breathing rate. As shown by the schematic 700a of FIG. 7A, a single filter 702 may be utilized for estimating heart rate and breathing rate together or estimating only one of heart rate and breathing rate. As shown by the schematic 700b of FIG. 7B, separate filters 704, 706 may obtain both heart rate and breathing rate.

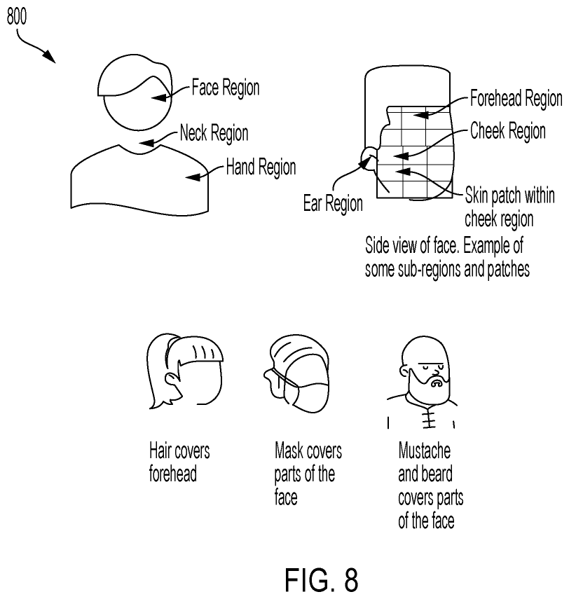

In general, skin may be exposed/visible from different regions like face, neck, hand, and the likes as shown by the chart 800 in FIG. 8. All skin pixels may be used for vital sign estimation in a combined manner, or the skin pixels may be divided into regions like face, neck, and hand for vital sign estimation. Face region for example may be further divided into sub-regions like forehead, cheek, nose, ear, and the like. Face may also be divided as small patches irrespective of forehead, cheek, and the like. The skin pixels whether processed region-wise or patch-wise or in a combined manner may be fused together appropriately for vital sign estimation. Practically certain regions may be covered/occluded, such as by hair, facial hair, or facial masks. The system may adaptively decide which regions to select.

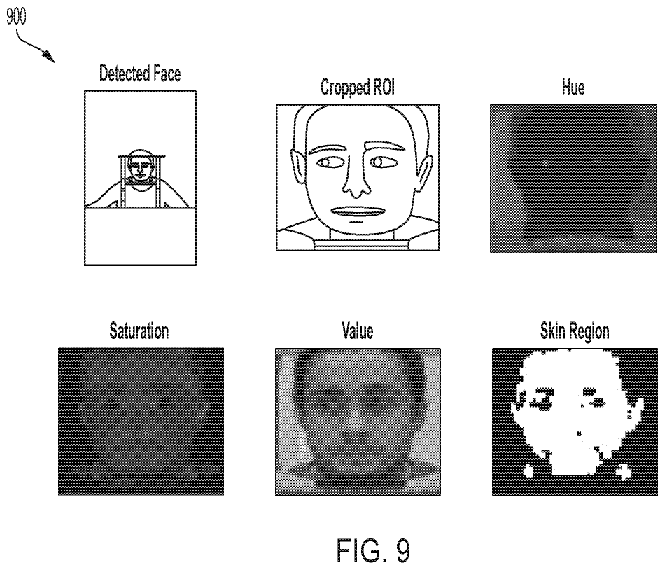

Referring to FIGS. 9-23, results have been calculated for frames 900 of a video from the publicly available dataset considered, such as for a resting condition and filtered using filters of order 6 and 186. Also, two window lengths have been considered for the algorithm to determine heart rate and heart rate variability, such as 60 and 128. Offline results with a window size of 128 may be the same as those with a window size of 60 since the same filter is used. The heart rate and heart rate variability estimation accuracies may vary. The results for filter order 6 are only given for both the window sizes.

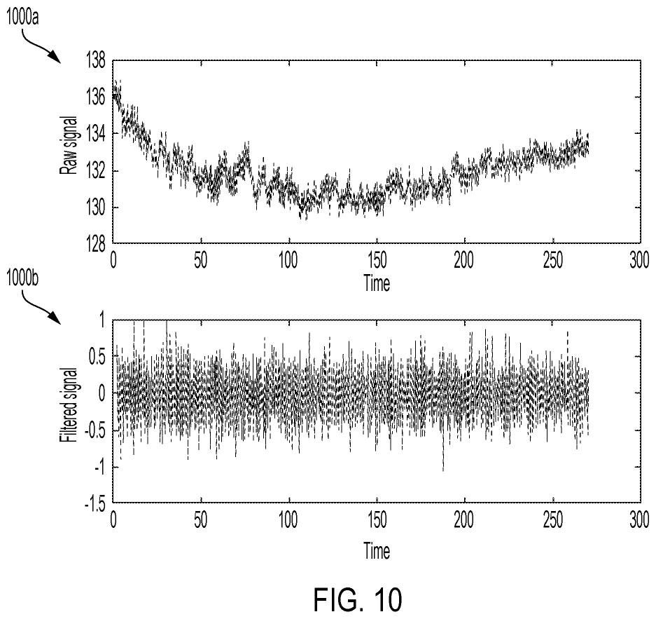

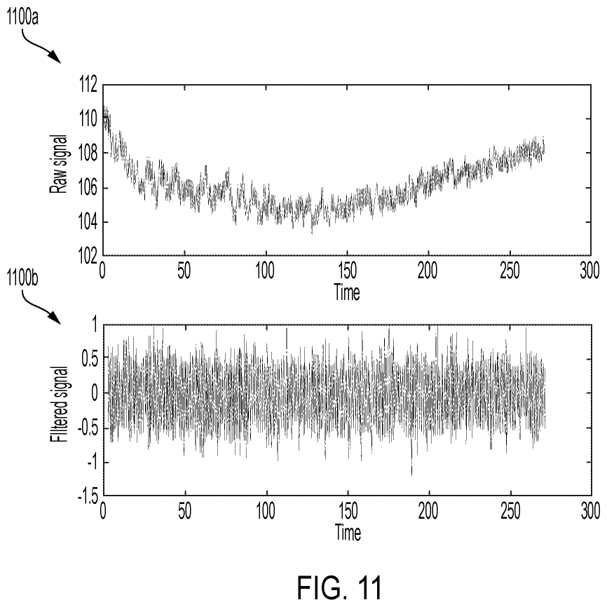

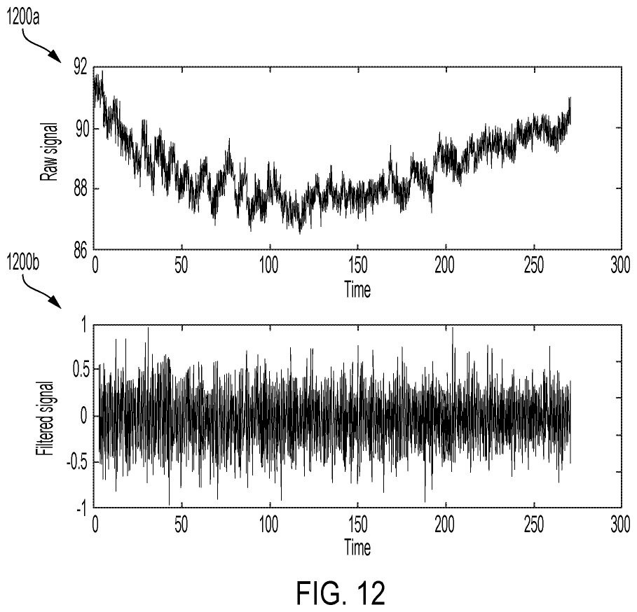

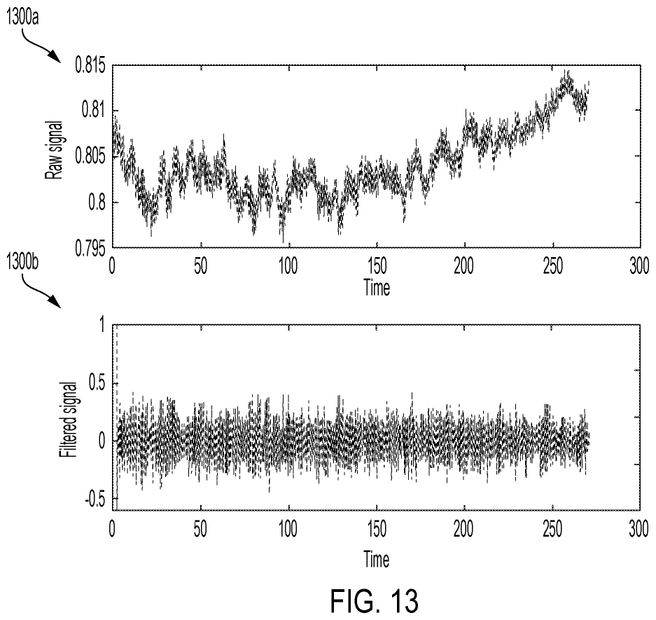

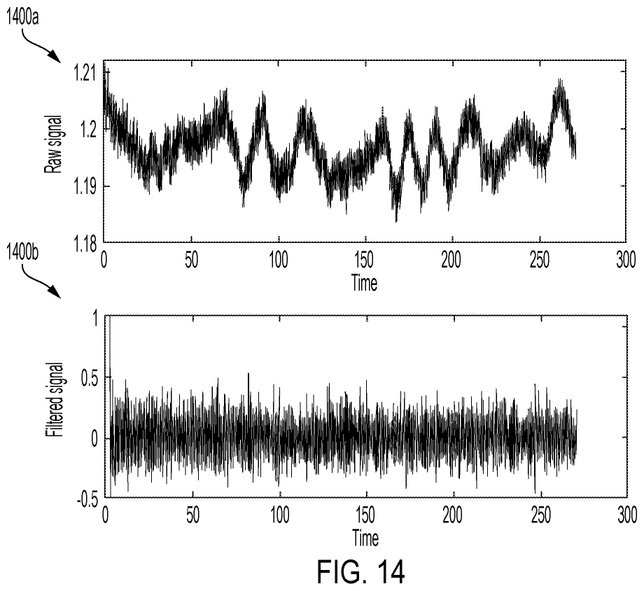

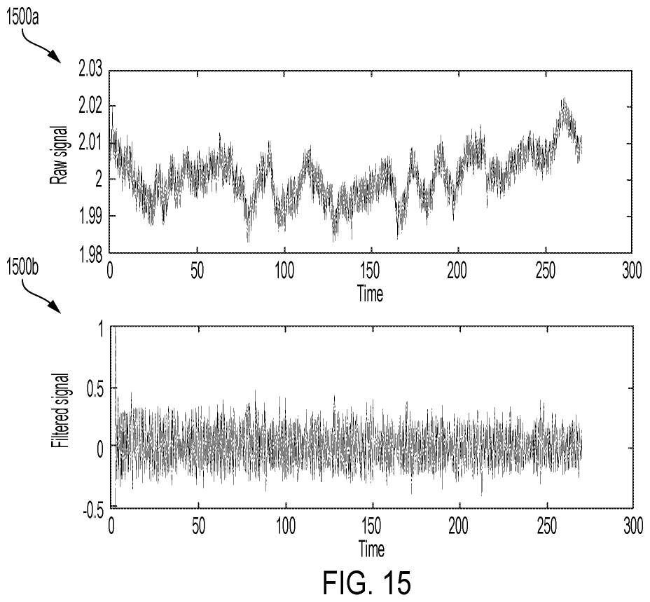

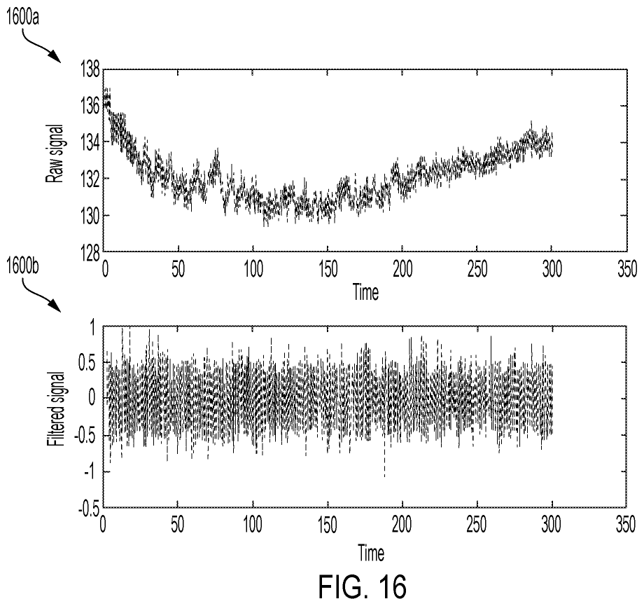

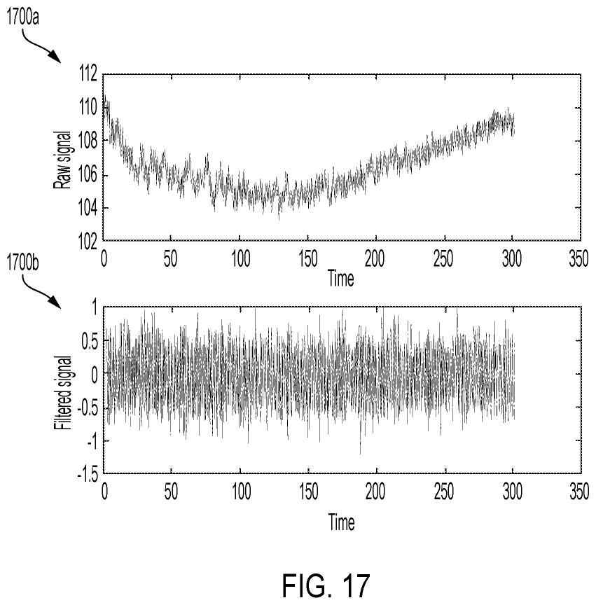

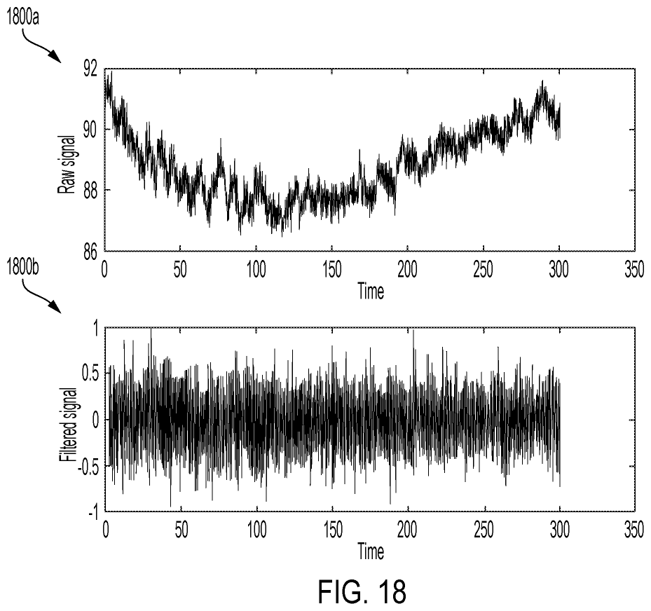

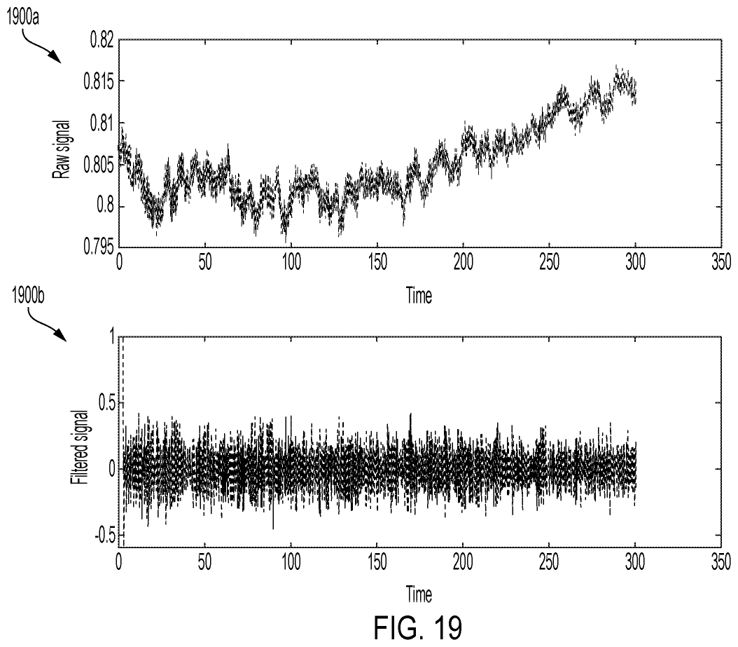

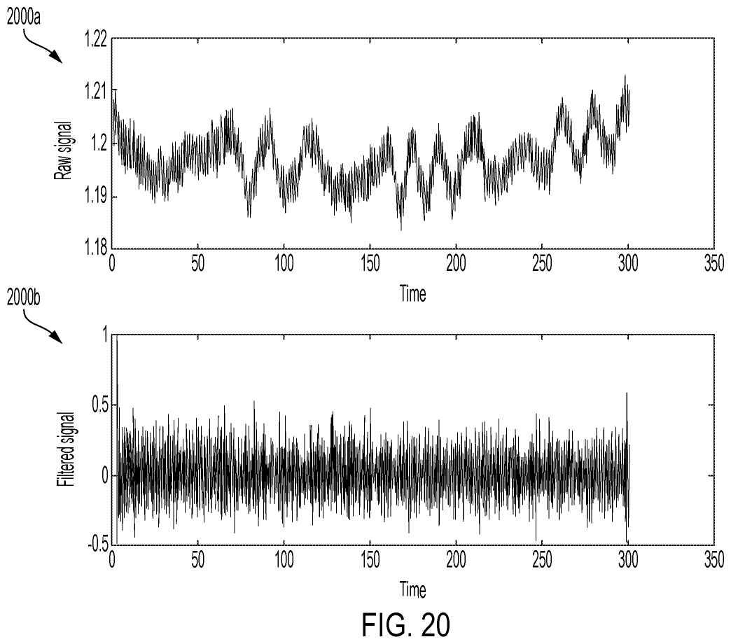

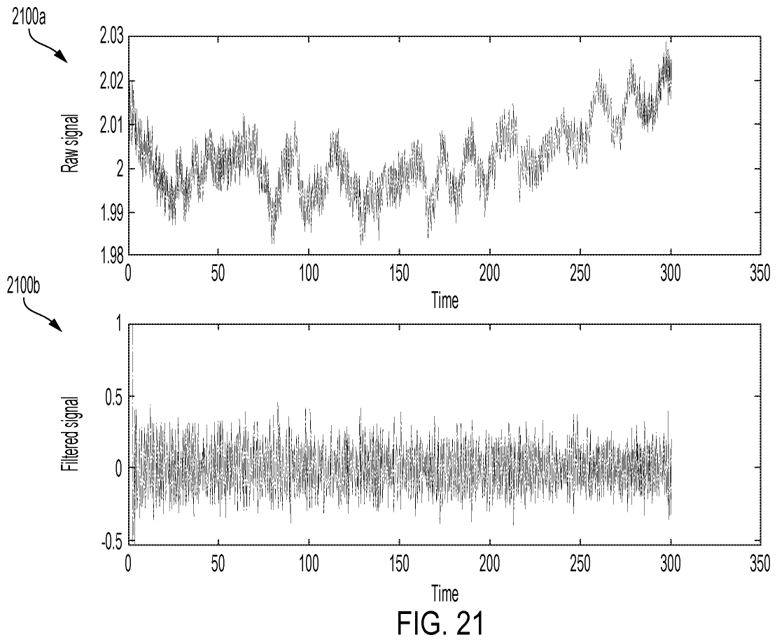

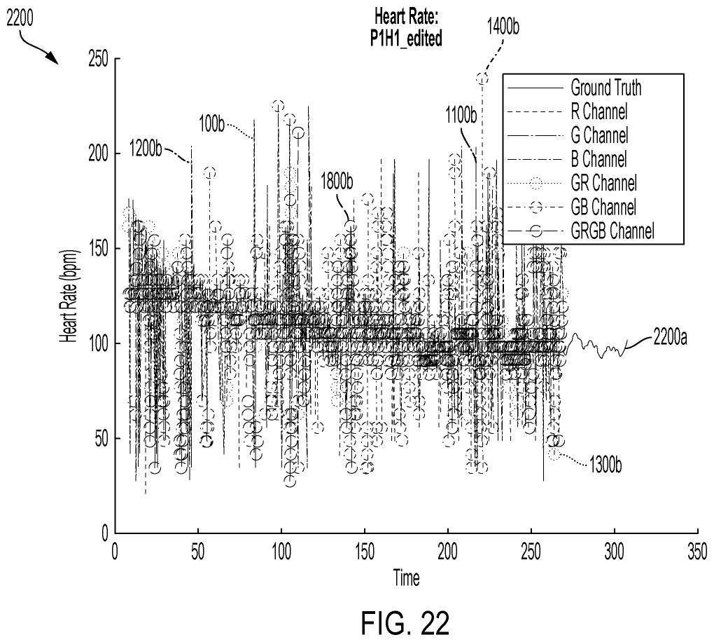

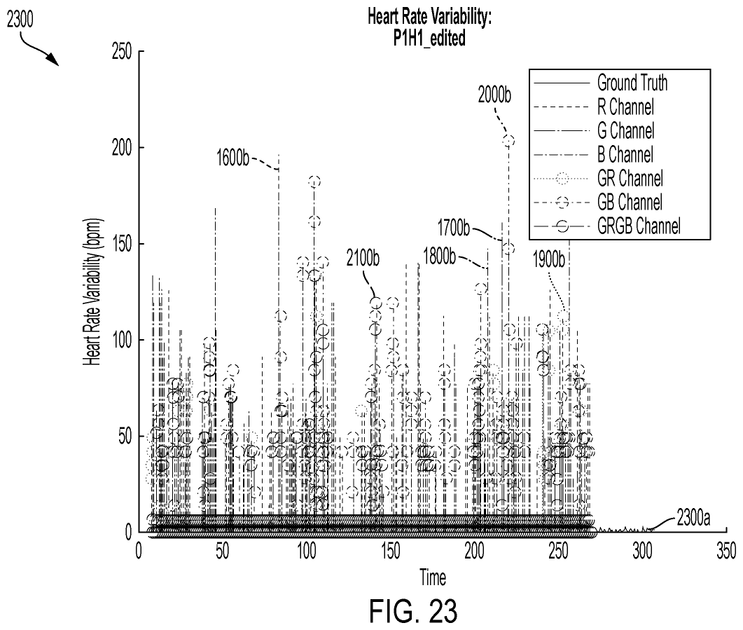

Graph 1000a represents raw red light signals and graph 1000b represents filtered red light signals with a window size of 60 (FIG. 10). Graph 1100a represents raw green light signals and graph 1100b represents filtered green light signals with a window size of 60 (FIG. 11). Graph 1200a represents raw blue light signals and graph 1200b represents filtered blue light signals with a window size of 60 (FIG. 12). Graph 1300a represents raw green-red light signals and graph 1300b represents filtered green-red light signals with a window size of 60 (FIG. 13). Graph 1400a represents raw green-blue light signals and graph 1400b represents filtered green-blue light signals with a window size of 60 (FIG. 14). Graph 1500a represents raw green-red-green-blue light signals and graph 1500b represents filtered green-red-green-blue light signals with a window size of 60 (FIG. 15). Graph 1600a represents raw red light signals and graph 1600b represents filtered red light signals with a window size of 128 (FIG. 16). Graph 1700a represents raw green light signals and graph 1700b represents filtered green light signals with a window size of 128 (FIG. 17). Graph 1800a represents raw blue light signals and graph 1800b represents filtered blue light signals with a window size of 128 (FIG. 18). Graph 1900a represents raw green-red light signals and graph 1900b represents filtered green-red light signals with a window size of 128 (FIG. 19). Graph 2000a represents raw green-blue light signals and graph 2000b represents filtered green-blue light signals with a window size of 128 (FIG. 20). Graph 2100a represents raw green-red-green-blue light signals and graph 2100b represents filtered green-red-green-blue light signals with a window size of 128 (FIG. 21). Graph 2200 represents filtered signals matched against the ground truth 2200a with a window size of 60 (FIG. 22). Graph 2300 represents filtered signals matched against the ground truth 2300a with a window size of 128 (FIG. 23).

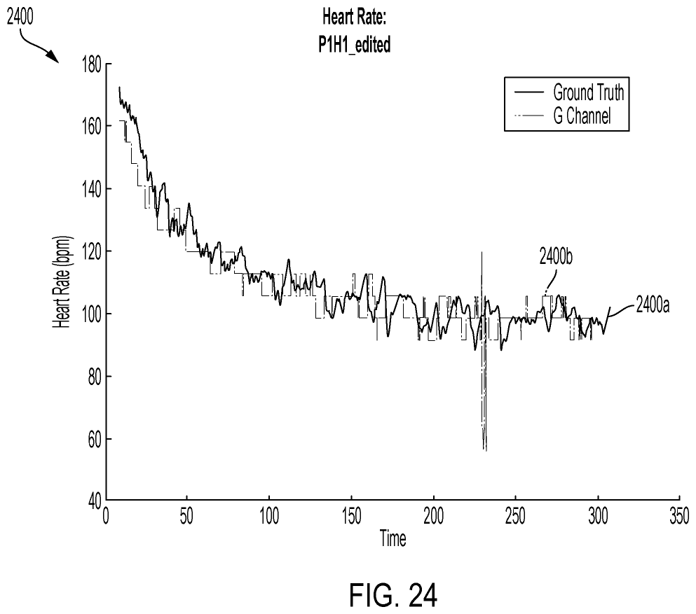

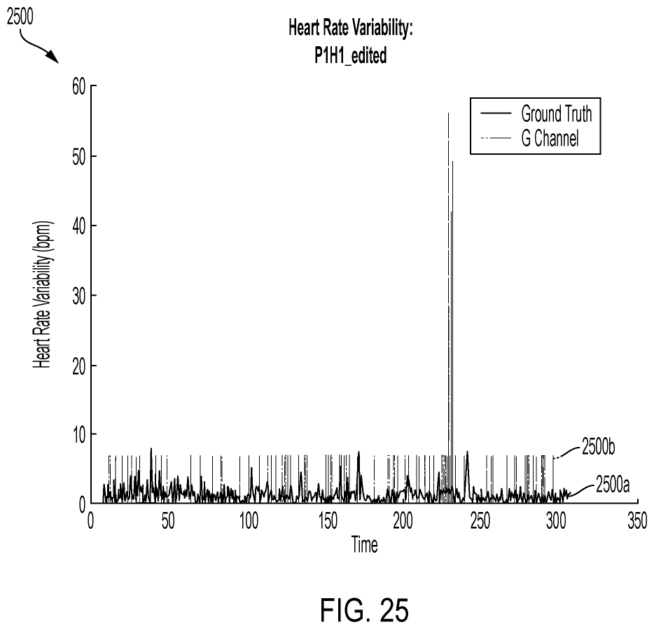

Referring to the graph 2400 of FIG. 24, example heart rate or ground truth data 2400a is compared to a heart rate 2400b determined based on the Green light channel and filtered using a filter order 6 with a buffer size of 128. Moreover, the graph 2500 of FIG. 25 shows example heart rate variability or ground truth data 2500a compared to a heart rate variability 2500b determined based on the Green light channel and filtered using a filter order 6 with a buffer size of 128.

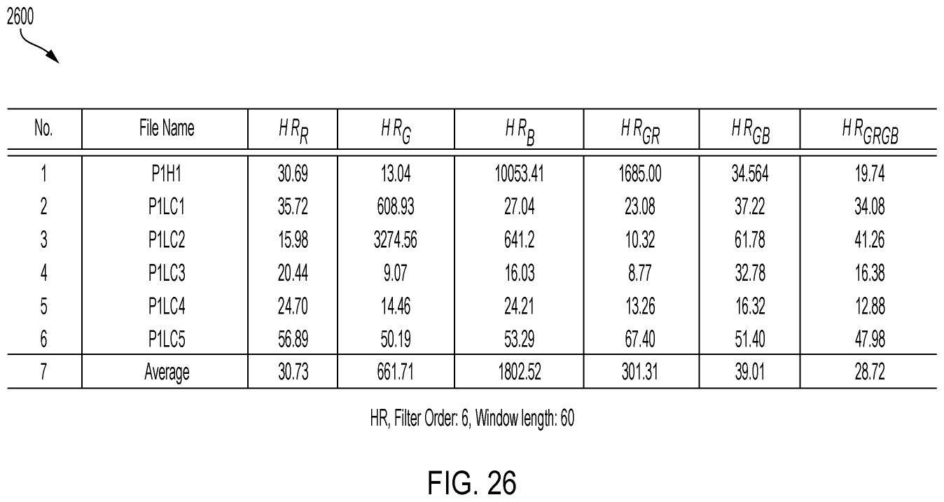

In the tables 2600, 2700, 2800, 2900, 3000, 3100, 3200, 3300, 3400 of FIGS. 26-34, P1H1, P1LC1, P1LC2, P1LC3, P1LC4, and P1LC5 refers to the video name in the database, HR stands for Heart Rate and HRV stands for Heart rate variability. The subscripts to these abbreviations refer to the signal component which could be Red, Green, Blue, Green-Red, Green-Blue and Green-Red-Green-Blue. The mean square error (MSE) is calculated between Ground truth given in the database and the results from the system processing.

Video P1H1 shows a high and descending heart rate that is recorded directly after physical exercise. The light temperature is 6,500 Kelvin and light intensity is 187 lux. Video P1LC1 has a light temperature of 6,500 Kelvin and light intensity of 5.2 lux. Video P1LC2 has a light temperature of 6,500 Kelvin and light intensity of 36 lux. Video P1LC3 has a light temperature of 6,500 Kelvin and light intensity of 187 lux. Video P1LC4 has a light temperature of 6,500 Kelvin and light intensity of 720 lux. Video P1LC5 has a light temperature of 6,500 Kelvin and light intensity of 2,720 lux.

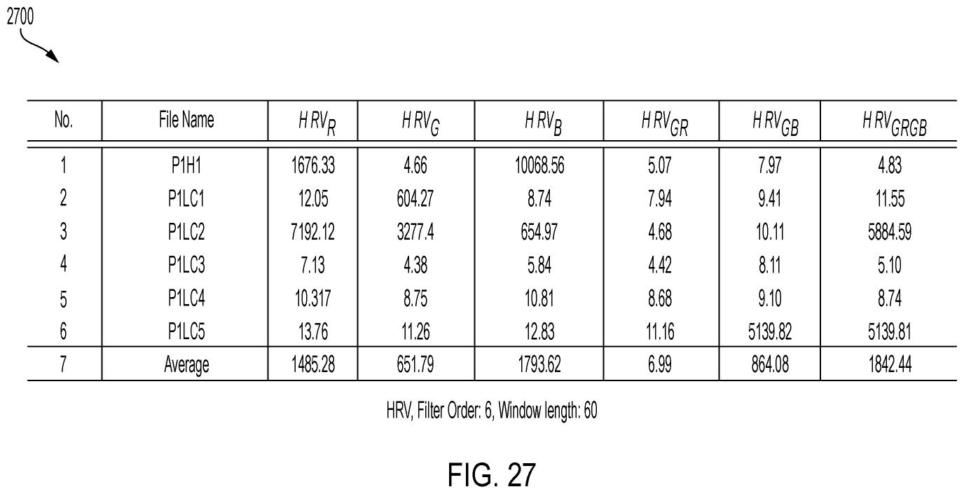

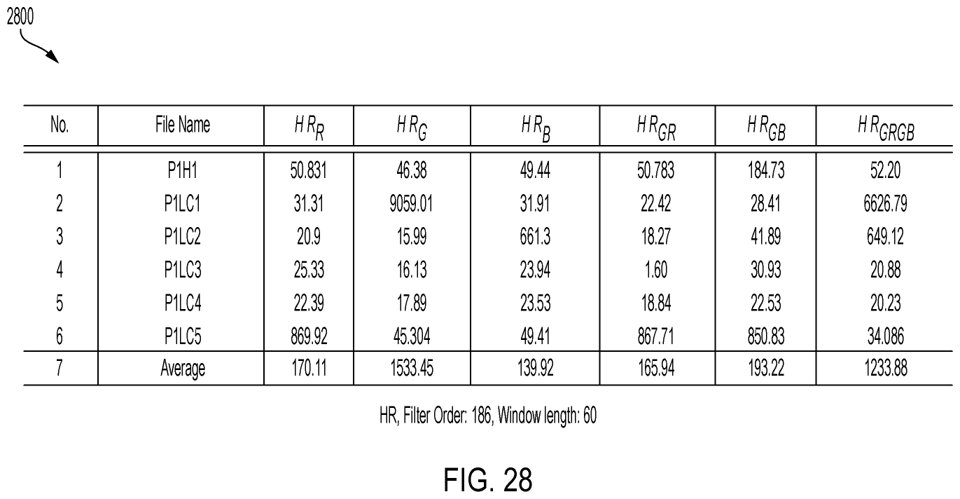

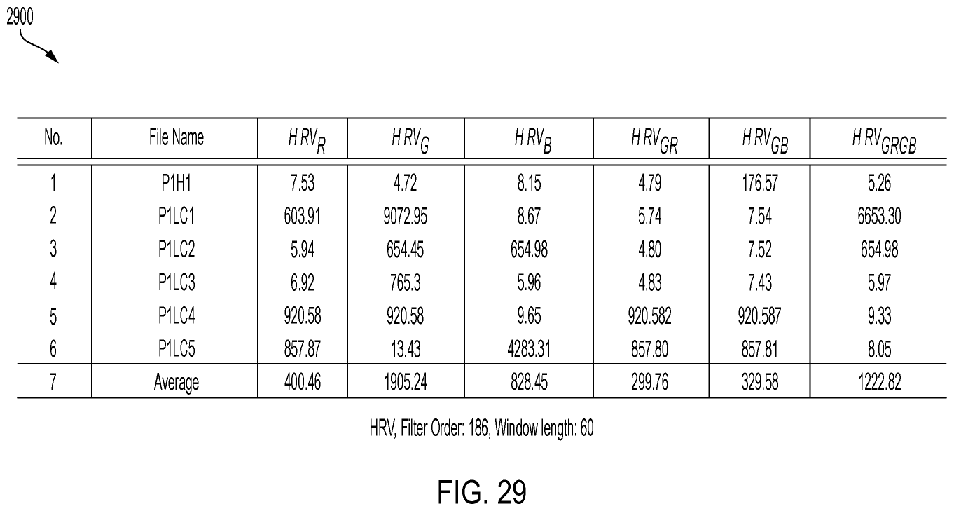

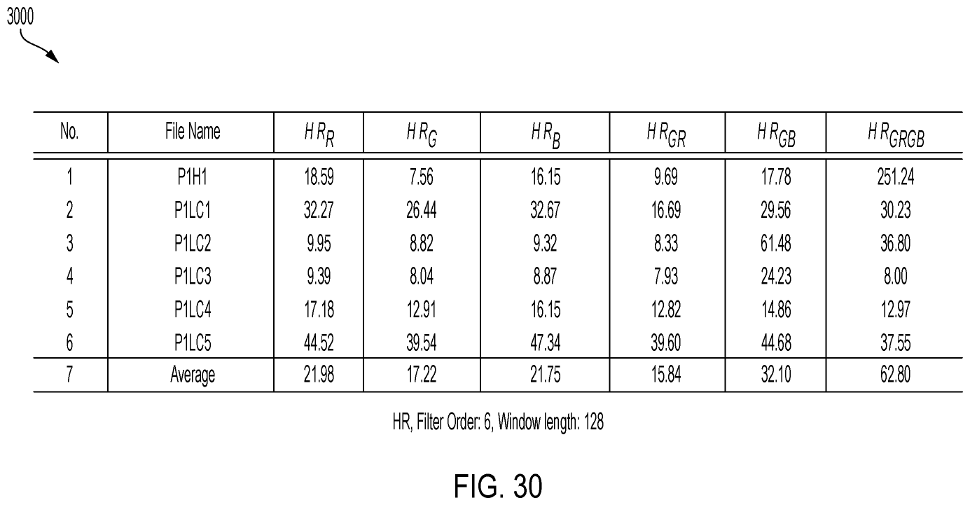

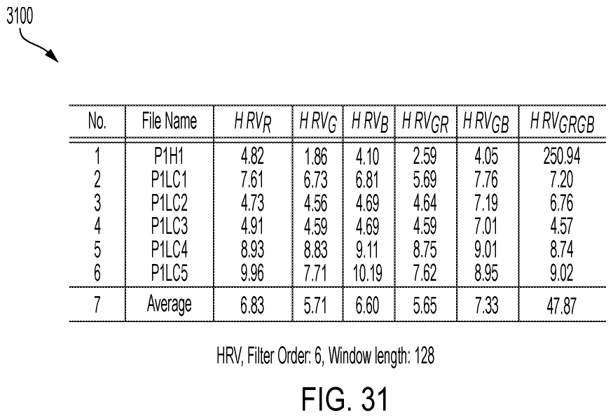

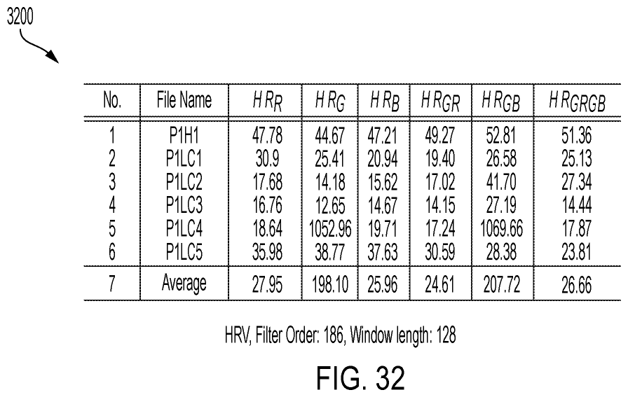

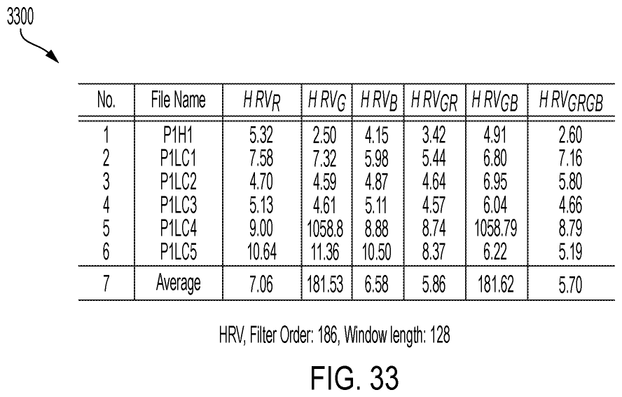

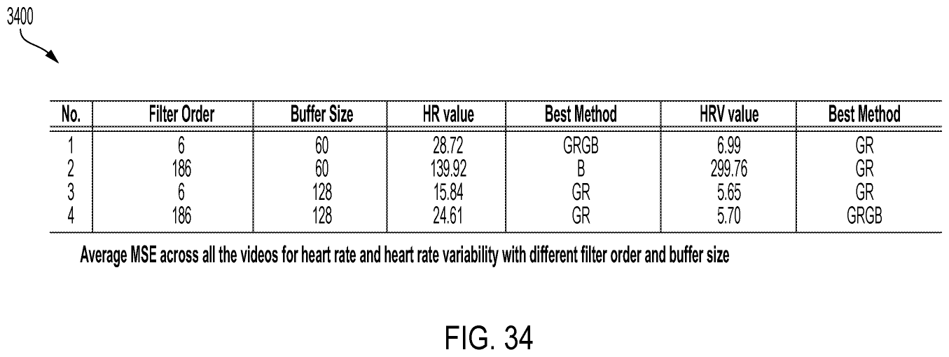

The chart 2600 of FIG. 26 represents heart rate determinations with a filter order 6 and window length of 60. The chart 2700 of FIG. 27 represents heart rate variability determinations with a filter order 6 and window length of 60. The chart 2800 of FIG. 28 represents heart rate determinations with a filter order 186 and window length of 60. The chart 2900 of FIG. 29 represents heart rate variability determinations with a filter order 186 and window length of 60. The chart 3000 of FIG. 30 represents heart rate determinations with a filter order 6 and window length of 128. The chart 3100 of FIG. 31 represents heart rate variability determinations with a filter order 6 and window length of 128. The chart 3200 of FIG. 32 represents heart rate determinations with a filter order 186 and window length of 128. The chart 3300 of FIG. 33 represents heart rate variability determinations with a filter order 186 and window length of 128. The chart 3400 of FIG. 34 shows the average MSE scores across all videos for heart rate and heart rate variability with different filter order and buffer size. The results indicate that the heart rate and heart rate variability may be best calculated (e.g., have the least Mean Square Error) from different channels based on the filter order and buffer or window size.

One aspect of the disclosure provides a vehicular cabin monitoring system that includes a camera disposed within an interior cabin of a vehicle equipped with the vehicular cabin monitoring system. The camera views within the interior cabin of the vehicle. An electronic control unit (ECU) includes electronic circuitry and associated software, including an image processor operable to process image data transferred to the ECU. The cabin monitoring system, via processing at the ECU of image data captured by the camera, monitors an occupant of the vehicle within the interior cabin of the vehicle. The cabin monitoring system determines a skin tone of the occupant of the vehicle. Based on the determined skin tone of the occupant of the vehicle, and responsive to detecting a change in complexion for the occupant of the vehicle, the system determines a health condition for the occupant of the vehicle. This aspect may include one or more of the following optional features.

In some implementations, based on detecting reflectance of color components of light incident at the occupant's face, the vehicular cabin monitoring system determines a red signal, a blue signal, and a green signal. The vehicular cabin monitoring system detects the change in facial complexion for the occupant of the vehicle based on a change in ratio between the red signal, the blue signal, and the green signal.

In some examples, the health condition includes at least one of (i) a heart rate of the occupant, (ii) a heart rate variance of the occupant and (ii) a breathing rate of the occupant. In some aspects, the camera is disposed at an interior rearview mirror assembly of the vehicle.

In some implementations, a profile associated with the occupant is stored in memory. The vehicular cabin monitoring system determines the skin tone of the occupant of the vehicle based at least in part on the profile associated with the occupant. In further implementations, the profile is retrieved from storage automatically based at least in part on performing facial recognition on the occupant. In further implementations, the profile includes a health history of the occupant. The vehicular cabin monitoring system performs an evasive maneuver based on a comparison of the determined health condition of the occupant to the health history of the occupant. In even further implementations, the evasive maneuver includes at least one of (i) sounding an alarm within the vehicle, (ii) transmitting a signal to emergency services, (iii) transmitting a signal to one or more preferred contacts of the occupant and (iv) controlling the vehicle to perform an automatic parking maneuver. The evasive maneuver may include generating an alert in other ways, such as generating an audio signal within the vehicle, generating a visual signal within the vehicle (e.g., at the infotainment screen or gauge cluster), or generating a haptic signal with in the vehicle (e.g., vibrating the steering wheel or driver seat). In further implementations, the determined health condition is added to the stored profile. The memory may be disposed at the vehicle or the memory may be disposed remote from the vehicle and in wireless communication with the vehicular cabin monitoring system at the vehicle.

In some examples, the system determines the skin tone of the occupant based at least in part on a signal from a system of the vehicle. For example, a driver monitoring system may transmit image data representative of the face of the driver for determining skin tone of the driver, or the driver monitoring system may transmit a signal indicative of an average skin tone of the driver. Then, the cabin monitoring system may monitor the complexion of the driver based on the signal from the DMS.

In some aspects, the vehicular cabin monitoring system determines the health condition episodically during operation of the vehicle, such as every 30 seconds, every 1 minute, every 5 minutes, every 10 minutes, and the like. In other aspects, the vehicular cabin monitoring system determines the health condition of the driver substantially continuously during operation of the vehicle. In other aspects, the system determines the health condition in response to an input, such as a determination that the occupant has entered the vehicle, based on determination that the vehicle has been turned on, based on determination of unusual or unexpected vehicle dynamics, based on determination that the driver is exhibiting drowsy or distracted behaviors, and/or based on determination that the vehicle has been involved in a collision.

In some implementations, the vehicular cabin monitoring system includes a plurality of cameras disposed within the interior cabin of the vehicle. The vehicular cabin monitoring system monitors the occupant of the vehicle via processing at the ECU of image data captured by the plurality of cameras. Each camera may capture image data of the same wavelength (e.g., visible light cameras or infrared cameras or near infrared cameras) or the cameras may capture image data of different wavelengths. Thus, the camera of the system may capture image data representative of visible light or near infrared light that is processed for monitoring the occupant, determining the skin tone of the occupant, and detecting changes in the complexion of the occupant.

The system may be calibrated for monitoring the occupant of the vehicle based on at least one of a model of the vehicle, a profile of the occupant, and a visible skin region of the occupant. For example, different models of vehicle may have different lighting conditions within the cabin of the vehicle and the system may be calibrated for detecting skin tones based on the lighting conditions within the cabin of the vehicle (e.g., including lighting provided by near infrared light illuminators within the cabin). Calibrating based on the profile of the occupant or a visible skin region of the occupant may indicate an average skin tone of the occupant so that the system may detect more minute changes in complexion. The system may be calibrated upon installation in the vehicle or adaptively (i.e., throughout the life of the system). The occupant may include a driver of the vehicle or a passenger of the vehicle.

In some examples, the determined skin tone of the occupant includes an average pixel value for portions of captured image data representative of skin of the occupant. Thus, detecting the change in complexion for the occupant of the vehicle may include detecting a change in pixel value for portions of captured image data representative of skin of the occupant.

The vehicular cabin monitoring system may utilize characteristics of the heart rate measurement systems and driver monitoring systems described in U.S. Pat. No. 12,539,857 and/or 11,433,906, which are hereby incorporated herein by reference in their entireties.

The system may utilize aspects of driver monitoring systems and/or head and face direction and position tracking systems and/or eye tracking systems and/or gesture recognition systems. Such head and face direction and/or position tracking systems and/or eye tracking systems and/or gesture recognition systems may utilize aspects of the systems described in U.S. Pat. Nos. 12,403,834; 12,393,089; 11,827,153; 11,780,372; 11,639,134; 11,582,425; 11,518,401; 10,958,830; 10,065,574; 10,017,114; 9,405,120 and/or 7,914,187, and/or U.S. Publication Nos. US-2025-0329174; US-2024-0383406; US-2024-0190456; US-2024-0168355; US-2022-0377219; US-2022-0254132; US-2022-0242438; US-2021-0323473; US-2021-0291739; US-2020-0320320; US-2020-0202151; US-2020-0143560; US-2019-0210615; US-2018-0231976; US-2018-0222414; US-2017-0274906; US-2017-0217367; US-2016-0209647; US-2016-0137126; US-2015-0352953; US-2015-0296135; US-2015-0294169; US-2015-0232030; US-2015-0092042; US-2015-0022664; US-2015-0015710; US-2015-0009010 and/or US-2014-0336876, and/or International Publication Nos. WO 2026/019985 and/or WO 2025/231198, which are all hereby incorporated herein by reference in their entireties.

The interior-viewing camera may be disposed at the mirror head of the interior rearview mirror assembly and moves together and in tandem with the mirror head when the driver of the vehicle adjusts the mirror head to adjust his or her rearward view. The interior-viewing camera may be disposed at a lower or chin region of the mirror head below the mirror reflective element of the mirror head, or the interior-viewing camera may be disposed behind the mirror reflective element and viewing through the mirror reflective element. Similarly, the light emitter may be disposed at the lower or chin region of the mirror head below the mirror reflective element of the mirror head (such as to one side or the other of the interior-viewing camera), or the light emitter may be disposed behind the mirror reflective element and emitting light that passes through the mirror reflective element. The ECU may be disposed at the mirror assembly (such as accommodated by the mirror head), or the ECU may be disposed elsewhere in the vehicle remote from the mirror assembly, whereby image data captured by the interior-viewing camera may be transferred to the ECU via a coaxial cable or other suitable communication line. Cabin monitoring or occupant detection may be achieved via processing at the ECU of image data captured by the interior-viewing camera. Optionally, cabin monitoring or occupant detection may be achieved in part via processing at the ECU of radar data captured by one or more interior-sensing radar sensors disposed within the vehicle and sensing the interior cabin of the vehicle.

The sensing system may utilize aspects of the systems described in U.S. Pat. Nos. 10,866,306; 9,954,955; 9,869,762; 9,753,121; 9,689,967; 9,599,702; 9,575,160; 9,146,898; 9,036,026; 8,027,029; 8,013,780; 7,408,627; 7,405,812; 7,379,163; 7,379,100; 7,375,803; 7,352,454; 7,340,077; 7,321,111; 7,310,431; 7,283,213; 7,212,663; 7,203,356; 7,176,438; 7,157,685; 7,053,357; 6,919,549; 6,906,793; 6,876,775; 6,710,770; 6,690,354; 6,678,039; 6,674,895 and/or 6,587,186, and/or U.S. Publication Nos. US-2019-0339382; US-2018-0231635; US-2018-0045812; US-2018-0015875; US-2017-0356994; US-2017-0315231; US-2017-0276788; US-2017-0254873; US-2017-0222311 and/or US-2010-0245066, which are hereby incorporated herein by reference in their entireties.

The radar sensors of the sensing system each comprise a plurality of transmitters that transmit radio signals via a plurality of antennas, a plurality of receivers that receive radio signals via the plurality of antennas, with the received radio signals being transmitted radio signals that are reflected from an object present in the field of sensing of the respective radar sensor. The system includes an ECU or control that includes a data processor for processing sensor data captured by the radar sensors. The ECU or sensing system may be part of a driving monitoring system of the vehicle or occupant monitoring system of the vehicle or cabin monitoring system of the vehicle or driving assist system of the vehicle, with the driving assist system controlling at least one function or feature of the vehicle (such as to provide autonomous driving control of the vehicle) responsive to processing of the data captured by the radar sensors.

The radar sensor or sensors may be disposed at the vehicle so as to sense exterior of the vehicle. For example, the radar sensor may comprise a front or forward-sensing radar sensor mounted at a grille or front bumper of the vehicle, such as for use with an automatic emergency braking system of the vehicle, an adaptive cruise control system of the vehicle, a collision avoidance system of the vehicle, etc., or the radar sensor may comprise a corner radar sensor disposed at a front corner or rear corner of the vehicle, such as for use with a surround vision system of the vehicle, or the radar sensor may comprise a blind spot monitoring radars disposed at a rear fender of the vehicle for monitoring sideward/rearward of the vehicle for a blind spot monitoring and alert system of the vehicle. Optionally, the radar sensor or sensors may be disposed within the vehicle so as to sense interior of the vehicle, such as for use with a cabin monitoring system of the vehicle or a driver monitoring system of the vehicle or an occupant detection or monitoring system of the vehicle. The radar sensing system may comprise multiple input multiple output (MIMO) radar sensors having multiple transmitting antennas and multiple receiving antennas.

The ECU may be operable to process data for at least one driving assist system of the vehicle. For example, the ECU may be operable to process data (such as image data captured by a forward-viewing camera of the vehicle that views forward of the vehicle through the windshield of the vehicle) for at least one selected from the group consisting of (i) a headlamp control system of the vehicle, (ii) a pedestrian detection system of the vehicle, (iii) a traffic sign recognition system of the vehicle, (iv) a collision avoidance system of the vehicle, (v) an emergency braking system of the vehicle, (vi) a lane departure warning system of the vehicle, (vii) a lane keep assist system of the vehicle, (viii) a blind spot monitoring system of the vehicle and (ix) an adaptive cruise control system of the vehicle. Optionally, the ECU may also or otherwise process radar data captured by a radar sensor of the vehicle or other data captured by other sensors of the vehicle (such as other cameras or radar sensors or such as one or more lidar sensors of the vehicle). Optionally, the ECU may process captured data for an autonomous control system of the vehicle that controls steering and/or braking and/or accelerating of the vehicle as the vehicle travels along the road.

Changes and modifications in the specifically described embodiments can be carried out without departing from the principles of the invention, which is intended to be limited only by the scope of the appended claims, as interpreted according to the principles of patent law including the doctrine of equivalents.

1. A vehicular cabin monitoring system, the vehicular cabin monitoring system comprising:
a camera disposed within an interior cabin of a vehicle equipped with the vehicular cabin monitoring system, wherein the camera views within the interior cabin of the vehicle;
an electronic control unit (ECU);
wherein image data captured by the camera is transferred to the ECU;
wherein the ECU comprises electronic circuitry and associated software, and wherein the electronic circuitry of the ECU comprises an image processor operable to process image data transferred to the ECU;
wherein the vehicular cabin monitoring system, via processing at the ECU of image data captured by the camera, monitors an occupant of the vehicle within the interior cabin of the vehicle;
wherein the vehicular cabin monitoring system determines a skin tone of the occupant of the vehicle; and
wherein the vehicular cabin monitoring system, based on the determined skin tone of the occupant of the vehicle, and responsive to detecting a change in complexion for the occupant of the vehicle, determines a health condition for the occupant of the vehicle.

2. The vehicular cabin monitoring system of claim 1, wherein, based on detecting reflectance of color components of light incident at the occupant's face, the vehicular cabin monitoring system determines a red signal, a blue signal, and a green signal, and wherein the vehicular cabin monitoring system detects the change in facial complexion for the occupant of the vehicle based on a change in ratio between the red signal, the blue signal, and the green signal.

3. The vehicular cabin monitoring system of claim 1, wherein the health condition comprises at least one selected from the group consisting of (i) a heart rate of the occupant, (ii) a heart rate variance of the occupant and (ii) a breathing rate of the occupant.

4. The vehicular cabin monitoring system of claim 1, wherein the camera is disposed at an interior rearview mirror assembly of the vehicle.

5. The vehicular cabin monitoring system of claim 1, wherein a profile associated with the occupant is stored in memory, and wherein the vehicular cabin monitoring system determines the skin tone of the occupant of the vehicle based at least in part on the profile associated with the occupant.

6. The vehicular cabin monitoring system of claim 5, wherein the profile is retrieved from storage automatically based at least in part on performing facial recognition on the occupant.

7. The vehicular cabin monitoring system of claim 5, wherein the profile comprises a health history of the occupant, and wherein the vehicular cabin monitoring system performs an evasive maneuver based on a comparison of the determined health condition of the occupant to the health history of the occupant.

8. The vehicular cabin monitoring system of claim 7, wherein the evasive maneuver comprises at least one selected from the group consisting of (i) sounding an alarm within the vehicle, (ii) transmitting a signal to emergency services, (iii) transmitting a signal to one or more preferred contacts of the occupant and (iv) controlling the vehicle to perform an automatic parking maneuver.

9. The vehicular cabin monitoring system of claim 5, wherein the determined health condition is added to the stored profile.

10. The vehicular cabin monitoring system of claim 1, wherein the vehicular cabin monitoring system determines the health condition episodically during operation of the vehicle.

11. The vehicular cabin monitoring system of claim 1, wherein the vehicular cabin monitoring system determines the health condition in response to an input.

12. The vehicular cabin monitoring system of claim 11, wherein the input comprises determination that the vehicle has been involved in a collision.

13. The vehicular cabin monitoring system of claim 1, wherein the vehicular cabin monitoring system comprises a plurality of cameras disposed within the interior cabin of the vehicle, and wherein the vehicular cabin monitoring system monitors the occupant of the vehicle via processing at the ECU of image data captured by the plurality of cameras.

14. The vehicular cabin monitoring system of claim 1, wherein the camera captures image data representative of visible light.

15. The vehicular cabin monitoring system of claim 1, wherein the camera captures image data representative of near infrared light.

16. The vehicular cabin monitoring system of claim 1, wherein the determined skin tone of the occupant comprises an average pixel value for portions of captured image data representative of skin of the occupant.

17. The vehicular cabin monitoring system of claim 16, wherein detecting the change in complexion for the occupant of the vehicle comprises detecting a change in pixel value for portions of captured image data representative of skin of the occupant.

18. A vehicular cabin monitoring system, the vehicular cabin monitoring system comprising:
a vehicular interior rearview mirror assembly comprises a mirror head adjustable about a mounting structure, wherein the mounting structure is configured to mount the vehicular interior rearview mirror assembly at an interior portion of an interior cabin of a vehicle;
wherein the mirror head accommodates a mirror reflective element;
wherein a camera is accommodated by the mirror head, and wherein the camera views within the interior cabin of the vehicle;
wherein the mirror reflective element and the camera move together and in tandem with the mirror head when, with the vehicular interior rearview mirror assembly mounted at the interior portion of the interior cabin of the vehicle, the mirror head is adjusted about the mounting structure to provide a rearward view for a driver of the vehicle provided by the mirror reflective element;
an electronic control unit (ECU);
wherein image data captured by the camera is transferred to the ECU;
wherein the ECU comprises electronic circuitry and associated software, and wherein the electronic circuitry of the ECU comprises an image processor operable to process image data transferred to the ECU;
wherein the vehicular cabin monitoring system, via processing at the ECU of image data captured by the camera, monitors a driver of the vehicle within the interior cabin of the vehicle;
wherein the vehicular cabin monitoring system determines a skin tone of the driver of the vehicle; and
wherein the vehicular cabin monitoring system, based on the determined skin tone of the driver of the vehicle, and responsive to detecting a change in complexion for the driver of the vehicle, determines a health condition for the driver of the vehicle;
wherein the determined skin tone of the driver comprises an average pixel value for portions of captured image data representative of skin of the driver, and wherein detecting the change in complexion for the driver of the vehicle comprises detecting a change in pixel value for portions of captured image data representative of skin of the driver;
wherein the health condition comprises at least one selected from the group consisting of (i) a heart rate of the driver, (ii) a heart rate variance of the driver and (ii) a breathing rate of the driver; and
wherein the vehicular cabin monitoring system, based on determining a change in the health condition for the driver, performs an evasive maneuver.

19. The vehicular cabin monitoring system of claim 18, wherein the evasive maneuver comprises at least one selected from the group consisting of (i) sounding an alarm within the vehicle, (ii) transmitting a signal to emergency services, (iii) transmitting a signal to one or more preferred contacts of the driver and (iv) controlling the vehicle to perform an automatic parking maneuver.

20. The vehicular cabin monitoring system of claim 18, wherein at least one selected from the group consisting of (i) the camera captures image data representative of visible light and (ii) the camera captures image data representative of near infrared light.
